(19) United States
(12) Patent Application Publication
(10) Pub. No.: US 2010/0305666 A1
(43) Pub. Date: Dec. 2, 2010

(54) INDUCTION DEVICE FOR PHOTODYNAMIC THERAPY AND DIAGNOSIS

(75) Inventors: Shyh Liang Lou, Hsinchu City (TW); Tzong Rong Ger, Taichung County (TW); Yih Chih Hsu, Taoyuan County (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: CHUNG YUAN CHRISTIAN UNIVERSITY, Chung Li (TW)

(21) Appl. No.: 12/675,896

(22) PCT Filed: Aug. 27, 2008
(86) PCT No.: PCT/CN2008/001538

§ 371 Date: Mar. 1, 2010

(30) Foreign Application Priority Data
Aug. 28, 2007 (CN) 200710148151.1

(51) Int. Cl. A61N 5/06

(52) U.S. Cl. 607/88

(57) ABSTRACT

An induction device for photodynamic therapy and diagnosis is used in a living body, and for activating a photochemical reaction of a photosensitive agent in the living body. The induction device comprises a transmitter, and a light irradiator implanted in the living body. The transmitter is used for emitting a radio signal to penetrate a superficial layer of the living body. When the light irradiator receives the radio signal, the light irradiator emits light by the driving signal, such that the photosensitive agent undergoes a photochemical reaction.

### BACKGROUND OF THE INVENTION

1. Field of the Invention

The present invention relates to an induction device, and more particularly to an induction device for photodynamic therapy and diagnosis.

2. Related Art

With the rapid development of medical technologies, photodynamic therapy (PDT) is an advanced technology, representing a breakthrough over conventional cancer tumor treatments. When used in combination with conventional surgical tumor removal technologies, the PDT is employed after the majority of the cancer tumor is removed, so as to eliminate the remaining tumor cells, thereby achieving an optimal treatment effect. With the advantages of doing little harm to the human body, preventing immunological dysfunction of the patient, promoting rapid recovery of the patient after the course of treatment, and being capable of selectively killing tumor cells without harming normal cells, the PDT has become one of the most attractive medical technologies in recent years.

During PDT, a photosensitive agent is firstly injected into or applied on the patient, and a period of time is given for the photosensitive agent to attach to tumor cells, and then a light irradiation device emits light of a particular wavelength to activate a photoactivation reaction of the photosensitive agent, so as to induce the toxicity of tumor cells, thereby eliminating the tumor cells.

Conventional PDT is limited by insufficient penetration of the light source of the photodynamic irradiation device, and thus is mostly used for the treatment of superficial organs of the human body, for example, diseases such as skin cancer and oral cancer, and the irradiation device directly irradiates the affected area of the human body, so as to achieve the treatment effect of eliminating the tumor cells.

In recent years, the PDT has found application to deep organ tissues of the human body. Taking the treatment of a brain tumor as an example, when a surgical operation is performed on the patient, a biocompatible material is implanted into the cranial cavity, and an optical fiber is led into the biocompatible material to perform light irradiation treatment on residual tumor cells in the cranial cavity, which overcomes the limit that conventional photodynamic devices cannot be used for the treatment of tumors in the cranial cavity.

However, for the patient, light irradiation performed on tissues in the cranial cavity through the optical fiber exposed outside the head is the invasive therapy, and a long course of treatment imposes a heavy burden to the patient both physically and psychologically, which not only causes considerable inconvenience to the daily life of the patient, but may also incur the risk of bacterial infection of the cranial cavity.

In order to alleviate the inconvenience and side effects caused by long-term treatment of the patient, the light source of the conventional photodynamic irradiation device mostly adopts a light emitting element with a high optical power, for example, a high-energy illuminant such as laser, so as to greatly reduce the time required by the light irradiation course, thereby alleviating the discomfort of the patient and reducing the risk of bacterial infection.

However, related research reports issued in recent years show that the treatment effect obtained by the high-optical power, short-term PDT method is not as good as the treatment effect obtained by continuously irradiating the affected area with a low optical power. Therefore, PDT in the prior art cannot provide safety and comfort for the patient during the course of treatment while achieving a desirable treatment effect at the same time.

### SUMMARY OF THE INVENTION

In view of the above problems, the present invention provides an induction device for photodynamic therapy and diagnosis, used in a living body, for activating a photochemical reaction of a photosensitive agent in the living body. The induction device comprises a transmitter, for emitting a radio signal to penetrate a superficial layer of the living body; and a light irradiator, implanted in the living body. The light irradiator comprises: a first induction coil, for receiving the radio signal; a control circuit, electrically connected to the first induction coil, for converting the radio signal into a driving signal; and a light emitting element, electrically connected to the control circuit, in which the light emitting element is driven by the driving signal to emit light, such that the photosensitive agent undergoes a photochemical reaction.

The present invention further provides an induction device for photodynamic therapy and diagnosis, so as to solve the problems that the photodynamic irradiation device in the prior art cannot be used for the diagnosis of diseases within the human body, that the conventional photodynamic irradiation device must be provided with an optical fiber line exposed outside the human body, causing discomfort and inconvenience to the patient during the course of treatment, and that conventional PDT cannot balance the physical and psychological burden on the patient with the desirable treatment effect.

The present invention further provides a light irradiator for photodynamic therapy and diagnosis, implanted in a living body. The light irradiator emits light through a radio signal penetrating a superficial layer of the living body, so as to activate a photochemical reaction of a photosensitive agent in the living body. The light irradiator comprises: a first induction coil, for receiving the radio signal; a control circuit, electrically connected to the first induction coil, for converting the radio signal into a driving signal; and a light emitting element, electrically connected to the control circuit, in which the light emitting element is driven by the driving signal to emit light, such that the photosensitive agent undergoes a photochemical reaction.

The induction device for photodynamic therapy and diagnosis of the present invention is used for the treatment of a living body, so as to activate a photochemical reaction of a photosensitive agent injected into the living body. The induction device comprises a transmitter and a light irradiator capable of being implanted into the living body. The light irradiator has a first induction coil, a control circuit electrically connected to the first induction coil, and a light emitting element electrically connected to the control circuit.

The transmitter is used for emitting a radio signal to penetrate the superficial layer of the living body. After the first induction coil of the light irradiator receives the radio signal, the control circuit converts the radio signal into a driving signal, so as to drive the light emitting element to emit light of a wavelength matching the photosensitive agent, such that the photosensitive agent undergoes a photochemical reaction.

The effect of the present invention lies in that, through wireless energy transfer, the transmitter located outside the living body electromagnetically induces the light irradiator implanted in the living body to emit light, so as to activate a reaction of the photosensitive agent, thereby performing diagnosis or treatment of the living body. The present invention not only alleviates various side effects, discomfort, and inconvenience in daily life caused by the conventional PDT device that uses wired transmission for treatment on the living body during the course of treatment, but also can balance the health and safety of the living body with the desirable treatment effect.

### BRIEF DESCRIPTION OF THE DRAWINGS

The present invention will become more fully understood from the detailed description given herein below for illustration only, and thus are not limitative of the present invention, and wherein:

FIG. 1A is a schematic plan view of an induction device according to the present invention;

FIG. 1B is a side view of an induction device applied on a human body according to the present invention;

FIG. 2 is a circuit block diagram of a transmitter according to the present invention;

FIG. 3A is a three-dimensional schematic view of a light irradiator according to the present invention;

FIG. 3B is a circuit block diagram of a light irradiator according to the present invention;

FIG. 3C is a circuit block diagram of a light irradiator having a secondary battery according to the present invention;

FIG. 4 is a measurement map of transmittance scattering rates of light of different optical powers passing through a fluid of a scattering element according to the present invention;

FIG. 5 is a circuit block diagram of an image capturing device according to the present invention;

FIG. 6A is a side view of an induction device applied to the diagnosis and therapy of tissues in the cranial cavity of a human body according to the present invention;

FIG. 6B is a side view of an induction device applied to the diagnosis and therapy of tissues in the oral cavity of a human body according to the present invention;

FIG. 6C is a side view of an induction device applied to the diagnosis and therapy of organs in the thoracic cavity of a human body according to the present invention;

FIG. 6D is a side view of an induction device applied to the diagnosis and therapy of organs in the abdominal cavity of a human body according to the present invention; and

FIG. 6E is a side view of an induction device applied to the diagnosis and therapy of organs in the pelvic cavity of a human body according to the present invention.

### DETAILED DESCRIPTION OF THE INVENTION

The induction device for photodynamic therapy and diagnosis of the present invention operates in a wireless energy transfer mode, and the wireless energy transfer mode comprises, but is not limited to, wireless transmission technologies such as radio frequency (RF) and microwave transmission. In the following detailed description of the present invention, RF transmission at about 1 MHz is taken as a preferred embodiment of the present invention. However, the accompanying drawings are merely provided for reference and description, but are not intended to limit the present invention.

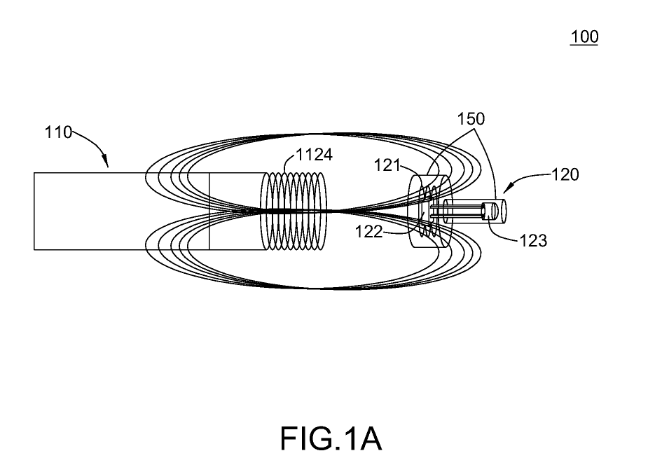

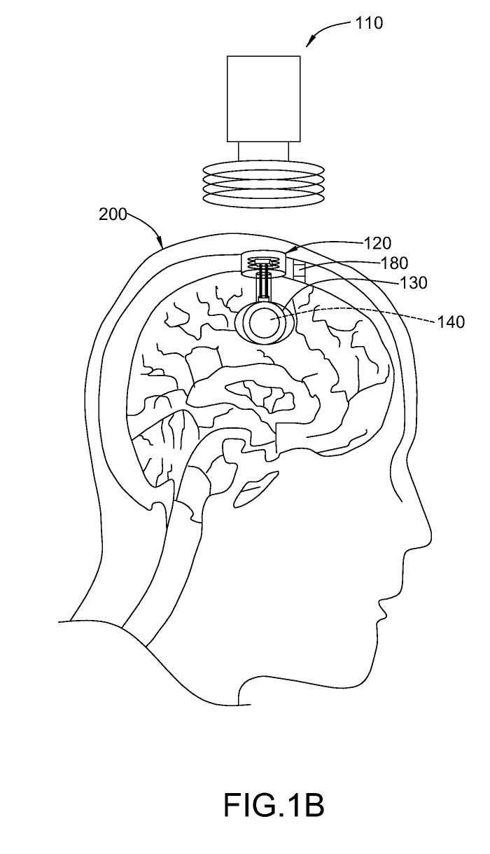

As shown in FIGS. 1A and 1B, an induction device 100 for photodynamic therapy and diagnosis of the present invention is used in a living body 200, for activating a photochemical reaction of a photosensitive agent (not shown) injected in the living body 200, so as to treat cells and tissues of the living body 200, for example, treatment of malignant cells, such as malignant tumors, melasma, skin whitening, scars, retinal macular degeneration, or development and marking of specific cells and tissues.

It should be noted that the induction device according to the embodiment of the present invention is described by taking the treatment of tumor cells in the human body as an example; however, persons skilled in the art may also apply the induction device to the diagnosis and therapy of various biologic species according to actual requirements in use, which is not limited to the embodiment of the present invention.

The induction device of the present invention comprises a transmitter 110 and a light irradiator 120 (as shown in FIG. 1B). The transmitter 110 has a second induction coil 1124, and is used for emitting an RF signal. The RF signal can be transferred into the living body 200 through a superficial layer of the living body 200, for example, skin, fat, muscle, and other superficial tissues.

The light irradiator 120 implanted in the living body 200 has a first induction coil 121, a control circuit 122 electrically connected to the first induction coil 121, and a light emitting element 123 electrically connected to the control circuit 122.

The first induction coil 121 is used for receiving the RF signal emitted from the transmitter 110, and the control circuit 122 converts the RF signal into a driving signal having a voltage, so as to generate a current. The light emitting element 123 may be a light source generator such as a light-emitting diode (LED) or a laser transmitter, and after receiving the current generated by the driving signal, the light emitting element 123 emits light of a particular wavelength matching the photosensitive agent, so as to activate a photochemical reaction of the photosensitive agent.

The embodiment of the present invention is described by taking a photosensitive agent such as 5-aminolevulinic acid (5-ALA) and a hematoporphyrin agent (for example, Photofrin) as an example; however, persons skilled in the art of PDT may use different photosensitive agents in combination with light matching the photosensitive agents for activation according to actual requirements in use, which is not limited to the embodiment of the present invention. A preferable activating wavelength for agents such as 5-ALA and Photofrin is about 630 nm, and a red light source emitted from the light emitting element 123 is used in combination, so as to activate a photochemical reaction of 5-ALA and Photofrin, thereby treating tumor cells of brain cancer, esophagus cancer, gastrointestinal cancer, bladder cancer, or lung cancer in the living body 200. With the advantages of low toxicity, rapid metabolism, and short time for protection against light of only 1 to 2 days, the photosensitive agent 5-ALA is widely applied in clinical treatment.

The reaction of the photosensitive agent cannot be activated unless the photosensitive agent receives energy of a certain intensity. Therefore, in the present invention, the treatment time during the treatment can be precisely controlled by controlling parameters such as the optical power of the light emitting element 123 or the light irradiation time, and discomfort and side effects caused to the living body 200 by excessively high optical power can be avoided.

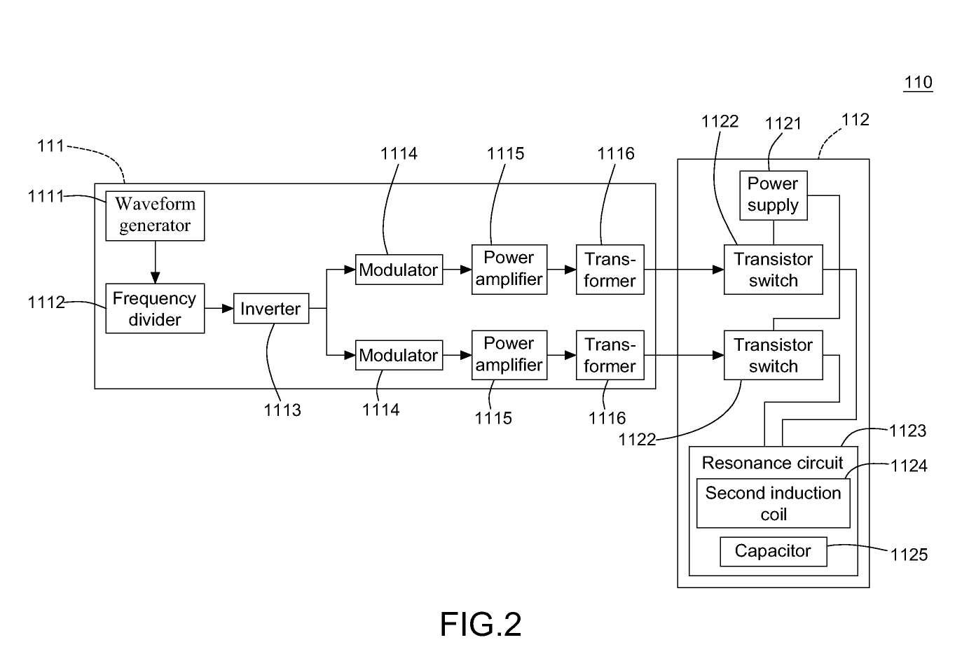

FIG. 2 is a circuit block diagram of a transmitter. As shown in FIG. 2, the transmitter 110 of the present invention comprises a driving circuit 111 and an oscillating circuit 112. The driving circuit 111 has a waveform generator 1111, a frequency divider 1112 electrically connected to the waveform generator 1111, an inverter 1113 electrically connected to the frequency divider 1112, two modulators 1114 electrically connected to the inverter 1113, two Class-D power amplifiers 1115 respectively electrically connected to the modulators 1114, and two transformers 1116 respectively electrically connected to the Class-D power amplifiers 1115.

The waveform generator 1111 is used for generating a square wave. The square wave is adjusted by the frequency divider 1112 to a particular range of carrier frequencies, and then the inverter 1113 transfers the adjusted square wave to the two modulators 1114, so as to respectively generate two opposite square wave signals having fixed wave width and amplitude. The square wave signals are transferred to the transformers 1116 through the gain effect of the power amplifiers, and converted by the two transformers 1116, such that the square wave signals are respectively output as control signals in the form of a sine wave and a cosine wave.

The oscillating circuit 112 of the transmitter 110 comprises a power supply 1121, two transistor switches 1122 electrically connected to the power supply 1121, and a resonance circuit 1123 electrically connected to the transistor switches 1122. The resonance circuit 1123 has a second induction coil 1124 electrically connected to the transistor switches 1122, and a capacitor 1125 electrically connected to the second induction coil 1124.

The transistor switches 1122 are used for respectively receiving the control signals of the transformers 1116, and each transistor switch 1122 performs an ON or OFF operation on the contrary to the other transistor switch 1122. The power supply 1121 continuously conducts a current to the resonance circuit 1123 according to the transistor switch 1122 in the ON state, and the second induction coil 1124 generates and emits a radio signal to the first induction coil 121 through charging and discharging functions of the capacitor 1125, so as to form magnetic field inductive coupling.

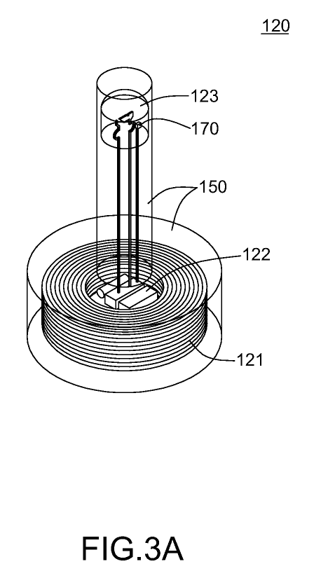

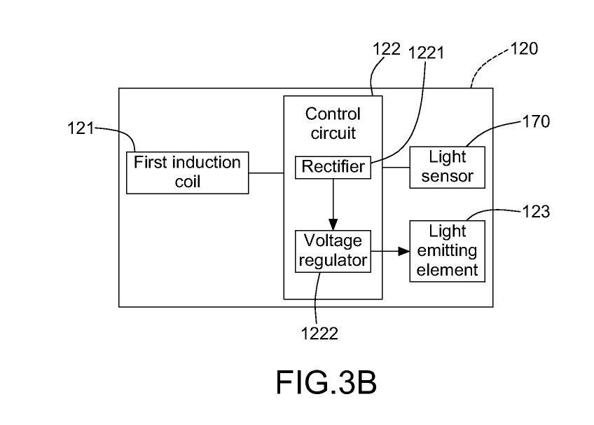

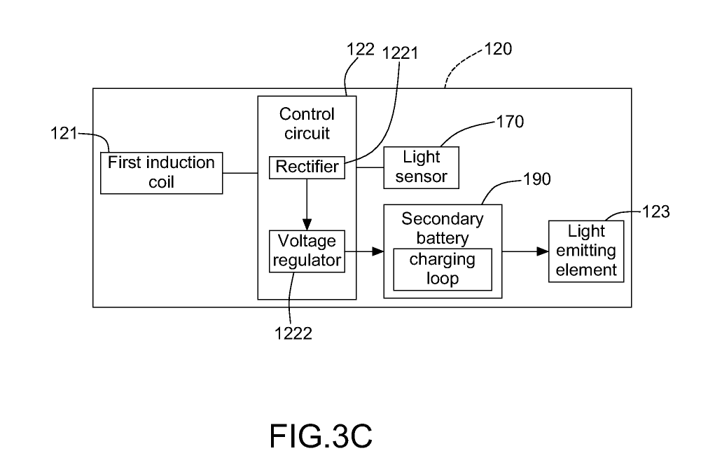

FIG. 3A is a three-dimensional schematic view of a light irradiator, and FIG. 3B is a circuit block diagram of the light irradiator. Referring to FIGS. 3A and 3B, the control circuit 122 of the light irradiator 120 has a rectifier 1221, and a voltage regulator 1222 electrically connected to the rectifier 1221 and the light emitting element 123. The control circuit 122 converts a magnetic radio signal received by the first induction coil 121 into an electric driving signal. The driving signal is converted by the rectifier 1221 from the original bidirectional oscillating signal into a unidirectional oscillating signal, and is maintained in a certain output value range by the voltage regulator 1222, so as to prevent the light emitting element 123 from being damaged by instable voltage of the driving signal. The rectifier 1221 of the present invention may be a rectifier of any form, for example, a half-wave rectifier, a full-wave rectifier, or a bridge rectifier, and since the technology is a design known to persons skilled in the art, the details will not be described herein again.

Still referring to FIG. 3A, a transparent light-transmissive wrapping material 150 is further wrapped outside the light irradiator 120, so as to completely wrap the first induction coil 121, the control circuit 122, and the light emitting element 123. The wrapping material 150 is made of a silica gel material or a glass material for biological purpose, and not only has good biocompatibility, but also has excellent electrical insulation and heat dissipation effects, so as to avoid the discomfort caused to the living body 200 by the light irradiator 120 implanted in the living body 200 due to excessive heat generated during operation.

Since the light irradiator 120 has good biocompatibility, the light irradiator 120 may be optionally taken out of the living body 200 after the course of treatment, or placed in the living body 200 for a long period of time so as to overcome the inconvenience and risk of another surgical operation.

As shown in FIG. 1B, the present invention further comprises a scattering element 130 coupled to the light emitting element 123. The scattering element 130 placed in the living body 200 has good biocompatibility, and will not cause an immunological rejection of the living body 200. The scattering element 130 of the present invention is a spherical material having an accommodation space therein, and a fluid 140 is filled inside the scattering element 130. The fluid 140 is, for example, a fluid having a high scattering rate such as air, saline, or an oil-water mixture. Here, the oil-water mixture may be lipofundin or intralipid. The fluid 140 is used for scattering the light.

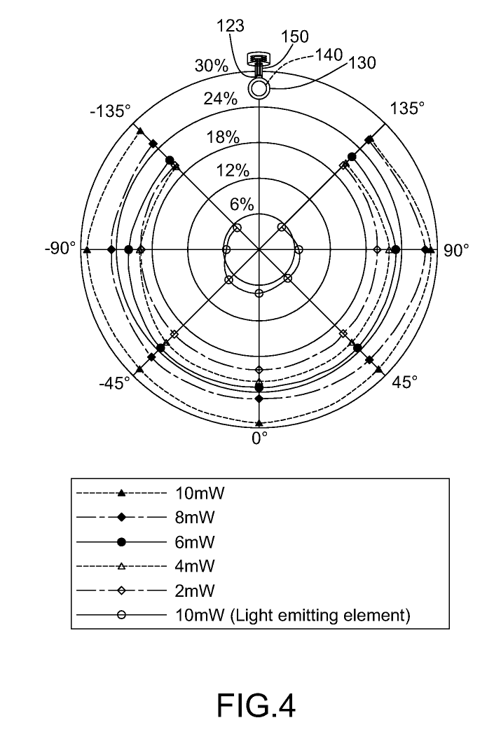

In the present invention, FIG. 4 uses the oil-water mixture lipofundin to describe this embodiment. Light of different powers emitted from the light emitting element 123 penetrates the fluid 140 (taking 0.12% lipofundin as an example) in the scattering element 130, and is then scattered to the outside, and the transmittance scattering rate of the light increases linearly as the optical power increases. An operator may select a suitable value for the optical power according to actual therapeutic requirements, so as to uniformly irradiate tissue cells in the living body 200, thereby achieving high efficiency and good PDT effect.

Moreover, FIG. 4 compares a difference in the transmittance scattering rate when the light emitting element 123 is disposed in the scattering element 130 and when the light emitting element 123 is coupled to a surface of the scattering element 130. As can be seen from measurement results, the transmittance scattering rate of the light emitting element 123 disposed in the scattering element 130 is far lower than the scattering rate of the light emitting element 123 coupled to the surface of the scattering element 130. Therefore, the configuration of the light emitting element 123 coupled to the surface of the scattering element 130 is described as a preferred embodiment of the present invention.

Moreover, in order to determine whether the light irradiator 120 implanted in the living body 200 really operates, as shown in FIGS. 3A and 3B, when the present invention is used for treatment, a light sensor 170 may be disposed in the light emitting element 123 to detect light refracted by a lens of the light emitting element 123 so as to calculate an intensity of light emitted from the light emitting element 123. The light sensor 170 is electrically connected to the control circuit 122 to detect an intensity of the light emitted from the light emitting element 123, and transfers an analog signal of the optical power to the control circuit 122 for digital demodulation of the signal. Then, the signal is transferred to an external demodulator (not shown) in a wireless transmission mode such as RF and microwave transmission, so as to obtain the current luminous intensity, and thus assist the practitioner to calculate the irradiation time required by the patient.

Alternatively, a light sensor (not shown) is disposed outside the living body 200 and adjacent to the light irradiator 120, so as to receive a current operating state of the light irradiator 120 returned by the light irradiator 120 in the form of a radio signal, for example, whether the light emitting element 123 emits light or the optical power of the light, such that the practitioner can know the treatment condition inside the living body 200.

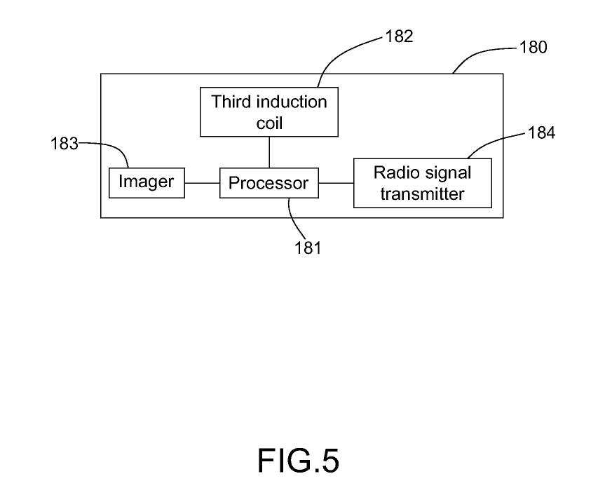

As shown in FIGS. 1B and 5, in order to observe the treatment condition inside the living body 200, an image capturing device 180 may be further disposed in the living body 200. The image capturing device 180 has a processor 181, a third induction coil 182 electrically connected to the processor 181, an imager 183, and a radio signal transmitter 184. The third induction coil 182 starts to operate upon receiving a radio signal emitted from the second induction coil 1124, and when the light irradiator 120 irradiates on the living body 200, the imager 183 detects a light intensity of the light passing through the living body 200, the processor 181 converts the light intensity into an output signal, and the radio signal transmitter 184 transfers the output signal to an external receiver (not shown) in a wireless transmission mode such as RF and microwave transmission, thereby obtaining the current treatment condition inside the living body 200.

In addition, in order to rapidly process the output signal generated by the image capturing device 180, two antennas (not shown) may be further disposed in the light irradiator 120 to respectively receive and transmit output signals, such that the output signals that are mainly image signals can be rapidly transmitted to the external receiver.

The image capturing device 180 may also be integrated in the light irradiator 120 without using the third induction coil 182, and the current generated by the control circuit 122 is shared to drive the imager 183 to operate, so that the device implanted in the living body 200 can be further simplified.

As shown in FIG. 3C, the light irradiator 120 of the present invention further comprises a secondary battery 190, electrically connected to the control circuit 122 and the light emitting element 123. The secondary battery 190 further has a charging-discharging loop 191. When the driving signal of the control circuit 122 is converted by the rectifier 1221 and the voltage regulator 1222 to charge the secondary battery 190, and the secondary battery 190 is activated by external wireless triggering to start or stop supplying electric energy to the light emitting element 123, the light irradiator 120 does not need to be always aligned with the external transmitter (not shown), so that the movement of the user is not limited, and continuous treatment is allowed. The starting or stopping of the electric energy output of the secondary battery 190 may be controlled by coupling signals of different frequencies, program codes in a microprocessor, demodulation of external signals by using carrier signal technologies, or other driving methods.

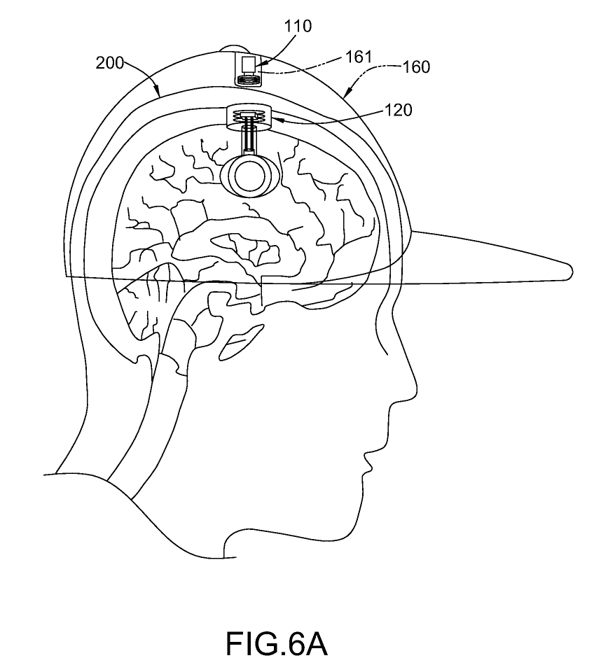

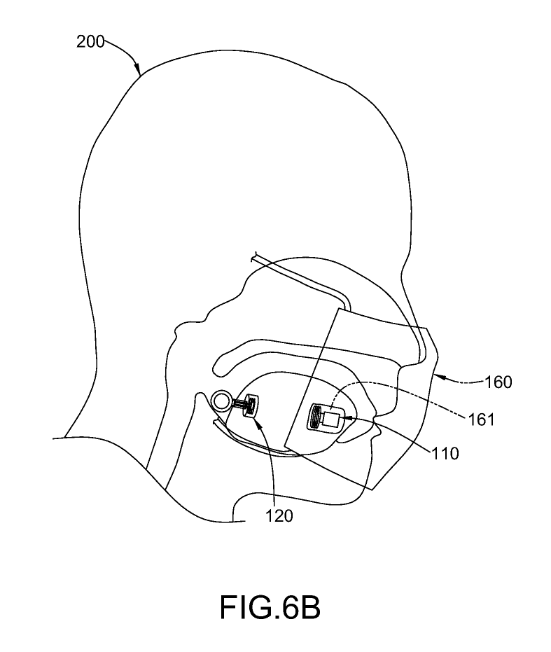

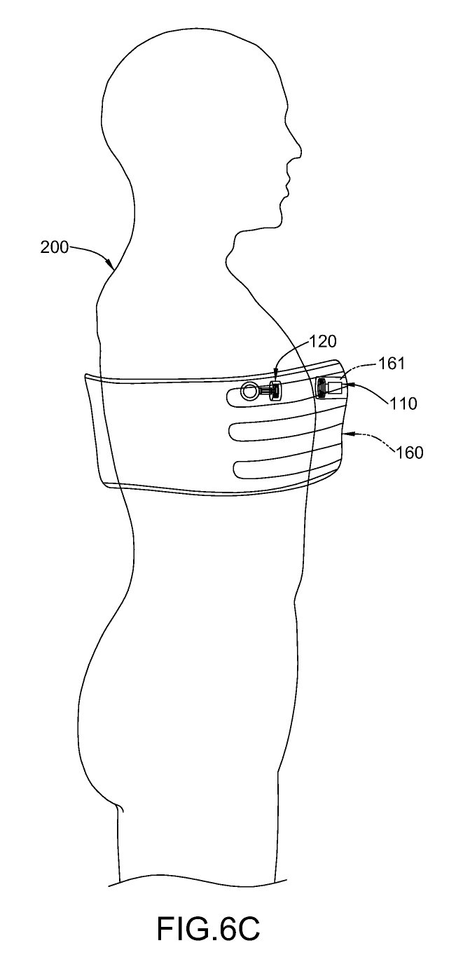

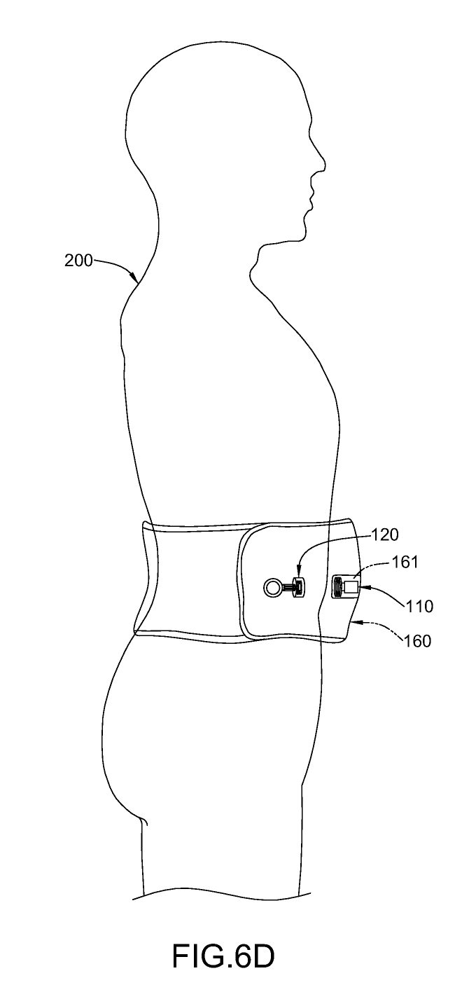

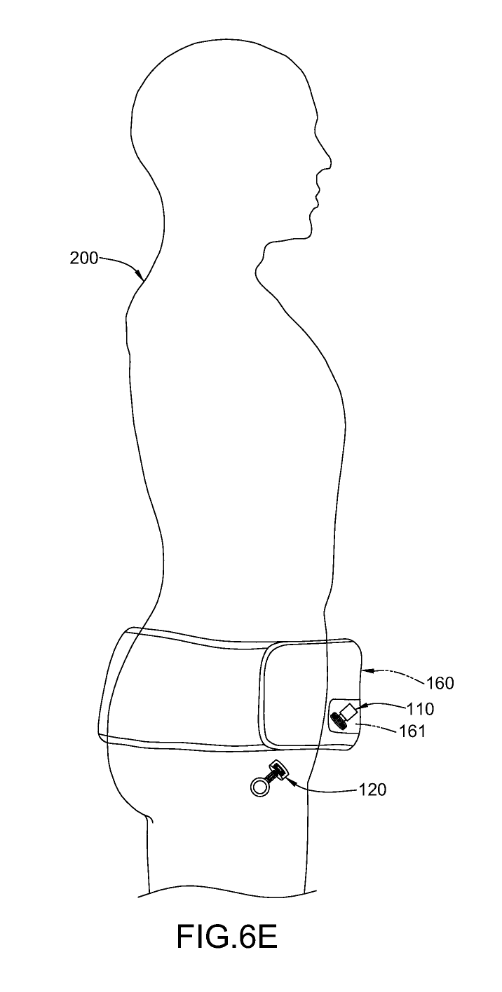

FIGS. 6A to 6E are schematic views of an induction device applied to the diagnosis and therapy of organs and tissues in different parts of a living body according to the present invention. The induction device 100 of the present invention may be applied to the therapy of various parts of the human body 200 (the living body), for example, the diagnosis and therapy of tumors of organs and tissues in the cranial cavity, oral cavity, thoracic cavity, abdominal cavity, pelvic cavity. The transmitter 110 of the present invention may be fixedly disposed at a position opposite to the light irradiator 120 in the human body 200 through a retaining piece 160 bound or attached to the human body 200, such that the transmitter 110 can perform RF transmission with the light irradiator 120, so as to implement the treatment.

The retaining piece 160 has an accommodation portion 161 for fixing the transmitter 110 therein. The retaining piece 160 may be designed as various forms such as a cap, respirator or girdle according to the part of the human body 200 to be treated, so that the retaining piece 160 can be bound or attached a position of the human body 200 corresponding to the light irradiator 120 conveniently.

Compared with the prior art, the transmitter located outside the living body electromagnetically induces the light irradiator implanted in the living body through radio signal transmission, and the light irradiator can emit light of a particular wavelength through electromagnetic conversion to activate a photochemical reaction of the matching photosensitive agent, thereby performing diagnosis or treatment of the living body. The present invention alleviates the inconvenience of a conventional PDT device that uses a wired transmission mode, and reduces the risk of bacterial infection of the human body.

Through the retaining piece for the transmitter of the present invention, the movement of the patient during the treatment is not limited by the transmitter, and the optical power or the light irradiation time of the light irradiator 120 can be precisely controlled, thereby alleviating discomfort caused to the living body during the treatment while achieving a desirable treatment effect.

What is claimed is:

1. An induction device for photodynamic therapy and diagnosis, used in a living body, for activating a photochemical reaction of a photosensitive agent in the living body, the induction device comprising:
a transmitter, for emitting a radio signal to penetrate a superficial layer of the living body; and
a light irradiator, implanted in the living body, and comprising:
a first induction coil, for receiving the radio signal;
a control circuit, electrically connected to the first induction coil, for converting the radio signal into a driving signal; and
a light emitting element, electrically connected to the control circuit, wherein the light emitting element is driven by the driving signal to emit light, such that the photosensitive agent undergoes a photochemical reaction.

2. The induction device for photodynamic therapy and diagnosis according to claim 1, wherein the transmitter comprises a driving circuit and an oscillating circuit, the driving circuit is used for generating a control signal, and the oscillating circuit is electrically connected to the driving circuit, so as to convert the control signal into the radio signal and emit the radio signal.

3. The induction device for photodynamic therapy and diagnosis according to claim 2, wherein the driving circuit further comprises a waveform generator, a frequency divider electrically connected to the waveform generator, an inverter electrically connected to the frequency divider, at least one modulator electrically connected to the inverter, at least one power amplifier electrically connected to the modulator, and at least one transformer electrically connected to the power amplifier, so as to generate the control signal.

4. The induction device for photodynamic therapy and diagnosis according to claim 2, wherein the oscillating circuit further comprises a power supply, at least one transistor switch electrically connected to the power supply, and a resonance circuit electrically connected to the transistor switch, and the transistor switch receives the control signal, so as to control the resonance circuit to emit the radio signal to the first induction coil.

5. The induction device for photodynamic therapy and diagnosis according to claim 4, wherein the resonance circuit comprises a second induction coil and a capacitor, the second induction coil is electrically connected to the transistor switch, the capacitor is electrically connected to the second induction coil, and the second induction coil emits the radio signal to the first induction coil through charging and discharging functions of the capacitor.

6. The induction device for photodynamic therapy and diagnosis according to claim 1, further comprising a scattering element coupled to the light emitting element, wherein the scattering element has an accommodation space and is filled with a fluid, so as to scatter the light.

7. The induction device for photodynamic therapy and diagnosis according to claim 6, wherein the fluid is selected from the group consisting of air, saline, and an oil-water mixture.

8. The induction device for photodynamic therapy and diagnosis according to claim 7, wherein the oil-water mixture is lipofundin or intralipid.

9. The induction device for photodynamic therapy and diagnosis according to claim 1, wherein the control circuit has a rectifier and a voltage regulator, the rectifier is used for receiving the driving signal, and the voltage regulator is electrically connected to the rectifier and the light emitting element, so as to rectify and regulate the driving signal.

10. The induction device for photodynamic therapy and diagnosis according to claim 1, further comprising a biocompatible wrapping material wrapped outside the light irradiator for electrical insulation and heat dissipation of the light irradiator.

11. The induction device for photodynamic therapy and diagnosis according to claim 10, wherein the wrapping material is a silica gel material or a glass material.

12. The induction device for photodynamic therapy and diagnosis according to claim 1, further comprising a retaining piece, wherein the retaining piece has an accommodation portion for fixing the transmitter on the retaining piece.

13. The induction device for photodynamic therapy and diagnosis according to claim 1, further comprising a light sensor, electrically connected to the control circuit, for detecting an intensity of the light when the light emitting element emits the light, and transferring the intensity to the control circuit.

14. The induction device for photodynamic therapy and diagnosis according to claim 1, further comprising an image capturing device, wherein the image capturing device comprises:
a processor;
a third induction coil, electrically connected to the processor, for receiving the radio signal, and driving the processor;
an imager, electrically connected to the processor, for detecting a light intensity of the light passing through the living body, wherein the processor converts the light intensity into an output signal; and
a radio signal transmitter, electrically connected to the processor, for transmitting the output signal.

15. The induction device for photodynamic therapy and diagnosis according to claim 1, wherein the light irradiator further comprises a secondary battery, electrically connected to the control circuit and the light emitting element, so as to supply electric energy to the light emitting element.

16. The induction device for photodynamic therapy and diagnosis according to claim 15, wherein the secondary battery further has a charging-discharging loop, and the charging-discharging loop charges the secondary battery through the driving signal of the control circuit.

17. A light irradiator for photodynamic therapy and diagnosis, implanted in a living body, wherein the light irradiator emits light through a radio signal penetrating a superficial layer of the living body, so as to activate a photochemical reaction of a photosensitive agent in the living body, the light irradiator comprising:
a first induction coil, for receiving the radio signal;
a control circuit, electrically connected to the first induction coil, for converting the radio signal into a driving signal; and
a light emitting element, electrically connected to the control circuit, wherein the light emitting element is driven by the driving signal to emit light, such that the photosensitive agent undergoes a photochemical reaction.

18. The light irradiator for photodynamic therapy and diagnosis according to claim 17, further comprising a scattering element coupled to the light emitting element, wherein the scattering element is filled with a fluid, so as to scatter the light.

19. The light irradiator for photodynamic therapy and diagnosis according to claim 17, wherein the control circuit has a rectifier and a voltage regulator, the rectifier is electrically connected to the first induction coil, and the voltage regulator is electrically connected to the rectifier and the light emitting element, so as to rectify and regulate the driving signal.

20. The light irradiator for photodynamic therapy and diagnosis according to claim 17, further comprising a biocompatible wrapping material wrapped outside the light irradiator for electrical insulation and heat dissipation of the light irradiator.

21. The light irradiator for photodynamic therapy and diagnosis according to claim 20, wherein the wrapping material is a silica gel material or a glass material.
